(19) United States
(12) Patent Application Publication
(10) Pub. No.: US 2011/0075791 A1
(43) Pub. Date: Mar. 31, 2011

(54) Radiographic image capturing apparatus, radiographic image capturing method, and position calculating method

(75) Inventors: Hiroki Nakayama, Kanagawa-ken (JP); Hajime Nakata, Kanagawa-ken (JP); Naoyuki Okada, Kanagawa-ken (JP); Takao Yoshida, Kanagawa-ken (JP)

(73) Assignee: FUJIFILM Corporation, Tokyo (JP)

(21) Appl. No.: 12/923,160

(22) Filed: Sep. 7, 2010

(30) Foreign Application Priority Data
Sep. 28, 2009 (JP) 2009-223138

(51) Int. Cl. A61B 6/00; G01N 23/00

(52) U.S. Cl. 378/4; 378/37

(57) ABSTRACT

A radiographic image capturing apparatus includes a radiation source that outputs radiation, a radiation detector for detecting the radiation and converting such radiation into a radiographic image, and a reference point, which is disposed in a removable manner between the radiation source and the radiation detector. The reference point is disposed at the position of a center of rotation of the radiation source, which is set between the radiation source and the radiation detector. By rotating the radiation source about the position of the center of rotation, the radiation source applies radiation with respect to the reference point from at least two different angles.

### CROSS-REFERENCE TO RELATED APPLICATION

This application is based upon and claims the benefit of priority from Japanese Patent Application No. 2009-223138 filed on Sep. 28, 2009, of which the contents are incorporated herein by reference.

### BACKGROUND OF THE INVENTION

1. Field of the Invention

The present invention relates to a radiographic image capturing apparatus and a radiographic image capturing method for applying radiation from a radiation source with respect to a reference point disposed between the radiation source and a radiation detector, and for detecting radiation by the radiation detector and converting the same into a radiographic image, as well as to a position calculating method for calculating a three dimensional position of the reference point based on the radiographic image.

2. Description of the Related Art

There have heretofore been developed biopsy apparatus for sampling tissue of a biopsy region (e.g., a lesion region in a subject's breast) contained within an object to be examined of the subject, and thoroughly examining the sampled tissue to perform a disease diagnosis. In this case, in order to reliably sample such tissue, it is essential that the three dimensional position of the biopsy region be accurately specified beforehand.

Consequently, a stereographic image capturing process is carried out in which, using the radiographic image capturing apparatus, radiation is applied respectively with respect to the examination object from a radiation source which is positioned at two different angles, radiation that has passed through the examination object is detected by a radiation detector, and two radiographic images are obtained. Based on the two radiographic images thus obtained, a calculation is performed to determine the three dimensional position of the biopsy region.

In this case, by rotating the radiation source about a central position of rotation, the radiation source can be moved to the aforementioned two angles. Further, the positional relationship between the focal position of the radiation source at the two angles and the radiation detector is regulated beforehand. However, in the case that the aforementioned angle (stereoscopic angle) becomes shifted, as a result of variances in assembly or manufacturing variances of the radiographic image capturing apparatus, the accuracy of the positional relationship becomes deteriorated, such that the three dimensional position of the biopsy region cannot be calculated with good and sufficient precision.

In response to this type of problem, in. Japanese Laid Open Patent Publication No. 2002-528220 (PCT), and in Japanese Laid-Open Patent Publication No. 2003-024321, there have been proposed stereographic image capturing processes, in which a reference point (marker) is disposed between a radiation source and a radiation detector, and radiation is applied from the radiation source with respect to the reference point as well as the examination object, whereby two radiographic images are obtained containing both the reference point and the examination object. In this case, based on the two radiographic images, the three dimensional position of the reference point and the three dimensional position of the biopsy region are calculated, such that the three dimensional position of the biopsy region can be corrected taking as a standard the three dimensional position of the reference point.

In this manner, in accordance with the disclosures of Japanese Laid-Open Patent Publication No. 2002-528220 (PCT) and Japanese Laid-Open Patent Publication No. 2003-024321, by simultaneously capturing stereographic images with respect to the reference point and the examination object, although the accuracy of the three dimensional position of the biopsy region may become deteriorated due to shifting in the stereoscopic angle as a result of variances in assembly or manufacturing variances of the radiographic image capturing apparatus, by correcting the three dimensional position of the biopsy region utilizing the three dimensional position of the reference point, degradation of the calculation result of the three dimensional position of the biopsy region can be suppressed.

Incidentally, while observing the two radiographic images obtained by stereographic image capturing, a doctor or radiological technician positions a biopsy device at the biopsy region from which tissue is to be sampled, or performs positioning to move the examination object to an appropriate position. Notwithstanding, if the two radiographic images are images in which the reference point is captured and included within the examination object, the existence of the reference point causes an obstacle to carrying out the aforementioned positioning procedure.

Although it might be considered to dispose the reference point at a position such that the reference paint would not be captured within the examination object, and to carry out stereographic image capturing in such a manner, in this case, the applied area (radiation field) of the radiation becomes enlarged (widened), and thus the examinee is exposed needlessly and excessively to radiation.

### SUMMARY OF THE INVENTION

It is an object of the present invention to enable correction of errors in the three dimensional position of a biopsy region caused by shifting of the stereoscopic angle, even though stereographic image capturing is not carried out simultaneously with respect to both a reference point and an object to be examined.

To accomplish the aforementioned object, a radiographic image capturing apparatus according to the present invention comprises a radiation source that outputs radiation, a radiation detector for detecting radiation and converting such radiation into a radiographic image, and a reference point, which is disposed in a removable manner between the radiation source and the radiation detector, wherein the reference point is disposed at a position of a center of rotation of the radiation source, which is set between the radiation source and the radiation detector, and wherein, by rotating the radiation source about the position of the center of rotation, the radiation source applies radiation with respect to the reference point from at least two different angles.

Further, a radiographic image capturing method according to the present invention comprises the steps of disposing a reference point in a removable manner on a position of an axis of rotation of a radiation source, which is set between the radiation source and a radiation detector, applying radiation by the radiation source with respect to the reference point from at least two different angles by rotating the radiation source about the position of the center of rotation, and detecting radiation by the radiation detector and converting such radiation into two radiographic images.

Furthermore, a position calculating method comprises the steps of disposing a reference point in a removable manner on a position of an axis of rotation of a radiation source, which is set between the radiation source and an image capturing base, applying radiation by the radiation source with respect to the reference point from at least two different angles by rotating the radiation source about the position of the center of rotation, detecting radiation by the radiation detector and converting such radiation into two radiographic images, and calculating a three dimensional position of the reference point by a position calculating unit based on the two radiographic images.

According to the present invention, because the reference point is disposed removably at the position of the center of rotation of the radiation source, when a stereographic image is taken with respect to the reference point, the reference point is disposed at the position of the center of rotation and stereographic image capturing may be performed, whereas, when a stereographic image is taken with respect to the biopsy region, the biopsy region is positioned and stereographic image capturing may be carried out after the reference point has been removed.

Owing thereto, radiographic images, which are obtained in each of the aforementioned stereographic image capturing processes, are made up of an image that contains the reference point therein, or alternatively, an image that contains only the biopsy region therein. Therefore, within each of these radiographic images, the reference point is not reflected in the object to be examined. Accordingly, a doctor or radiological technician while observing two radiographic images in which the object to be examined is imaged, a biopsy apparatus can easily be positioned on a biopsy region from which tissue is to be sampled, or positioning can easily be performed to move the object to be examined to an appropriate position.

Further, since the reference point is not present when a stereographic image is taken with respect to the examination object, the applied area (radiation field) of the radiation can be limited to within a minimum necessary range, and thus needless and excessive exposure of the examinee to such radiation can be avoided.

Further, a three dimensional position of the reference point is calculated based on two radiographic images obtained by carrying out stereographic image capturing with respect to the reference point, while on the other hand, a three dimensional position of the biopsy region inside the examination object is calculated based on two radiographic images obtained by carrying out stereographic image capturing with respect to the examination object.

Accordingly, even if there are errors in the three dimensional position of the biopsy region caused by shifting of the stereoscopic angle, the three dimensional position of the biopsy region can be corrected using the three dimensional position of the reference point.

In this manner, compared to the disclosures of Japanese Laid-Open Patent Publication No. 2002-528220 (PCT) and Japanese Laid-Open Patent Publication No. 2003-024321, with the present invention, the number of times that stereographic image capturing is carried out is increased by one. However, even though such stereographic image capturing is not performed simultaneously with respect to the reference point and the examination object, any errors in the three dimensional position of the biopsy region caused by shifting of the stereoscopic angle can easily be corrected.

In addition, the radiographic image capturing apparatus further comprises an image capturing base accommodating the radiation detector therein, wherein the reference point is disposed on a jig, which is arranged removably on the image capturing base.

Owing thereto, setting of the reference point at the position of the center of rotation can easily be carried out.

In this case, the radiographic image capturing apparatus further comprises a compression plate, which is displaceable toward the image capturing base to compress and secure an object to be examined of a subject on the image capturing base in case that the object to be examined is positioned on the image capturing base. In a state in which the jig is arranged on the image capturing base, and after the radiation source has applied radiation with respect to the reference point from the two angles, the jig is removed from the image capturing base. Then, in a state in which the jig has been removed from the image capturing base and the object to be examined has been compressed and secured between the compression plate and the image capturing base, the radiation source applies radiation with respect to the object to be examined from the two angles.

Owing thereto, assuming that the object to be examined is the breast of a subject and the biopsy region is a lesion within the breast, errors in the three dimensional position of the lesion area can be corrected effectively.

Additionally, assuming that, in front view, the position of the center of rotation and the position of the reference point are disposed at a predetermined position within the object to be examined, which is compressed and secured between the compression plate and the image capturing base, errors in the three dimensional position of a biopsy region caused by shifting of the stereoscopic angle can accurately be corrected.

Further, even in the case that, in plan view, the reference point is disposed at a center position inside the object to be examined, which is compressed and secured between the compression plate and the image capturing base, errors in the three dimensional position of a biopsy region caused by shifting of the stereoscopic angle can be corrected accurately.

Preferably, the radiographic image capturing apparatus further comprises a memory unit for storing two radiographic images obtained by the radiation detector by applying radiation with respect to the reference point, and also storing two radiographic images obtained by the radiation detector by applying radiation with respect to the object to be examined, which has been compressed and secured, and a position calculating unit for calculating a three dimensional position of the reference point and a three dimensional position of a biopsy region inside the object to be examined based on the radiographic images stored in the memory unit. The position calculating unit corrects the three dimensional position of the biopsy region inside the object to be examined using the three dimensional position of the reference point.

Owing thereto, the three dimensional position of the biopsy region can be corrected with better efficiency.

Further, preferably, in case that the jig is arranged on the image capturing base, an auxiliary reference point is disposed on the jig, wherein the auxiliary reference point is arranged on a vertical axis, which is orthogonal to the radiation detector and passes through the reference point and the position of the center of rotation. The radiation source applies radiation with respect to the reference point and the auxiliary reference point from the two angles in case that the jig is arranged on the image capturing base. Then, the memory unit stores two radiographic images obtained by the radiation detector by applying radiation with respect to the reference point and the auxiliary reference point. The position calculating unit calculates a three dimensional position of the reference point, a three dimensional position of the auxiliary reference point, and a three dimensional position of the biopsy region based on the radiographic images stored in the memory unit, and concerning the three dimensional position of the biopsy region, the position calculating unit corrects a position in a direction along the radiation detector based on the calculated three dimensional position of the reference point and the calculated three dimensional position of the auxiliary reference point.

Owing thereto, the three dimensional position of the biopsy region can be corrected with even greater precision.

The above and other objects, features and advantages of the present invention will become more apparent from the following description when taken in conjunction with the accompanying drawings in which a preferred embodiment of the present invention is shown by way of illustrative example.

### BRIEF DESCRIPTION OF THE DRAWINGS

FIG. 1 is a perspective view of a mammography apparatus according to an embodiment of the present invention;

FIG. 2 is a partial side view of the mammography apparatus shown in FIG. 1;

FIG. 3 is a perspective view showing a state in which a jig used for calibration is arranged on an image capturing base;

FIG. 4 is a side view showing application of radiation under a condition in which the jig of FIG. 3 is arranged on the image capturing base;

FIG. 5 is a frontal view showing stereographic image capturing under a condition in which the jig of FIG. 3 is arranged on the image capturing base;

FIG. 6 is a plan view showing a condition in which the jig of FIG. 3 is arranged on the image capturing base;

FIG. 7 is an explanatory drawing concerning positioning of a reference point;

FIG. 8 is an explanatory drawing concerning positioning of a reference point;

FIG. 9 is a perspective view showing a case in which another jig is arranged on the image capturing base;

FIG. 10 is a side view showing a condition in which the jig of FIG. 9 is arranged on the image capturing base;

FIG. 11 is a block diagram of the mammography apparatus shown in FIG. 1; and

FIG. 12 is a flowchart for explaining an operation sequence of the mammography apparatus.

### DESCRIPTION OF THE PREFERRED EMBODIMENTS

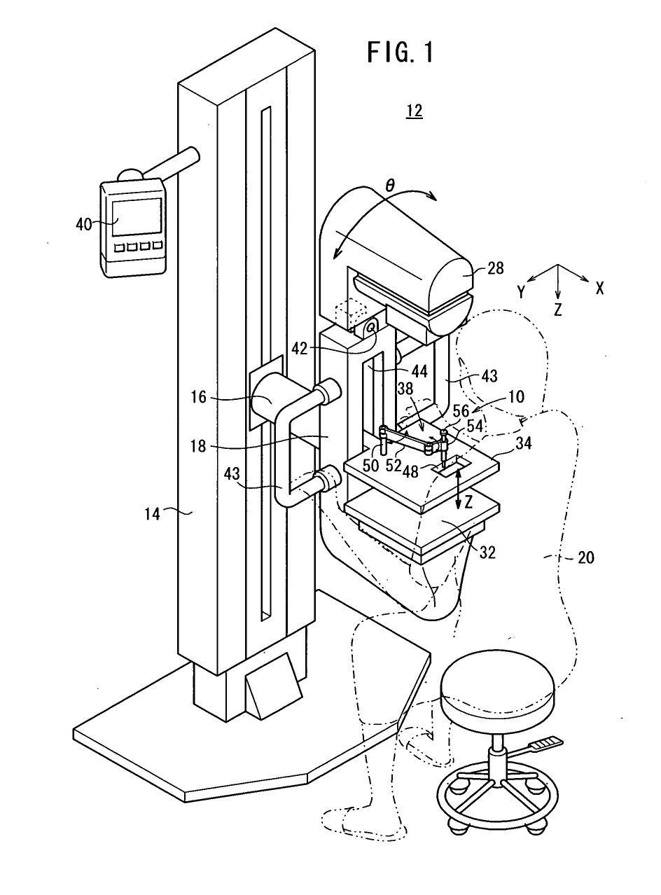

A radiographic image capturing apparatus according to a preferred embodiment of the present invention will be described below with reference to FIGS. 1 to 12, in relation to a radiographic image capturing method and position calculating method carried out by the radiographic image capturing apparatus.

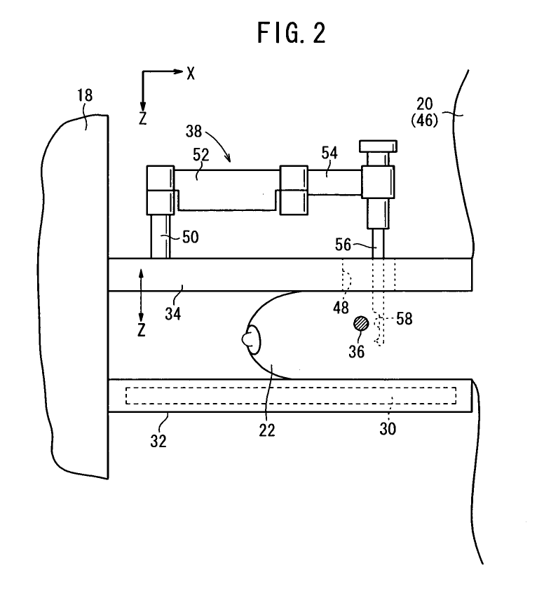

The basic structure of a mammography apparatus (radiographic image capturing apparatus, mammography image capturing apparatus) 12 according to an embodiment of the present invention, which incorporates a biopsy apparatus 10 therein, will be described below with reference to FIGS. 1 and 2.

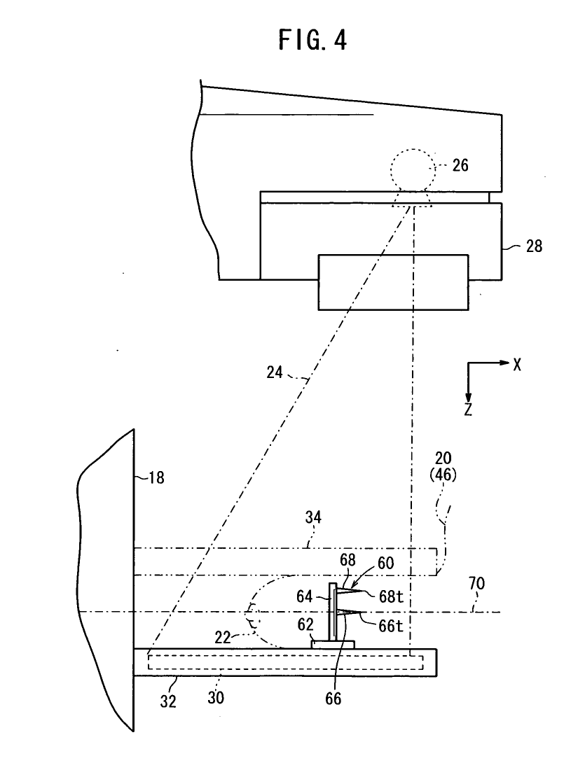

The mammography apparatus 12 essentially includes an upstanding base 14, a vertical arm 18 fixed to the distal end of a swing shaft 16 disposed substantially centrally on the base 14, a radiation source housing unit 28 fixed to an upper end of the arm 18 and housing therein a radiation source 26 for applying radiation 24 (see FIG. 4) to a breast 22, which defines a mass to be examined of an examinee (subject) 20, an image capturing base 32 mounted on a lower end of the arm 18 and housing therein a solid-state detector (radiation detector) 30 for detecting radiation 24 that has passed through the breast 22, a compression plate 34 for compressing and holding the breast 22 against the image capturing base 32, and a biopsy hand assembly 38 mounted on the compression plate 34 for removing a tissue sample from a biopsy region 36 of the breast 22.

As shown in FIGS. 1 and 2, the mammography apparatus 12 applies radiation 24 to the breast 22 of the examinee 20 and a sample tissue is removed from the biopsy region 36, while the breast 22 of the examinee 20, who is in a sitting position, is compressed and secured between the compression plate 34 and the image capturing base 32. Further, a display control panel 40 is connected to the base 14 for displaying image capturing conditions representing an image capturing region of the examinee 20, ID information of the examinee 20, etc., and for enabling setting of such items of information, as necessary.

When the arm 18, to which the radiation source housing unit 28 and the image capturing base 32 are secured, is angularly moved about the swing shaft 16, the direction of the radiation source housing unit 28 and the image capturing base 32 with respect to the breast 22 of the examinee 20 can be adjusted. The radiation source housing unit 28 is operatively coupled to the arm 18 by a hinge 42, and can be turned about the hinge 42 in directions indicated by the arrow θ independently of the image capturing base 32.

The arm 18 has a groove 44 defined vertically in a side (front side) thereof, which faces toward the examinee 20 in the direction indicated by the arrow X. The groove 44 extends along the direction indicated by the arrow Z. Handles 43 are mounted on respective sides of the arm 18, which face away from each other along the direction indicated by the arrow Y. The handles 43 may be gripped by the examinee 20. The compression plate 34 has a proximal end inserted into the groove 44 and held in interfitting engagement with a non-illustrated mount. The compression plate 34, which is thus coupled to the arm 18, is disposed between the radiation source housing unit 28 and the image capturing base 32. The compression plate 34 is vertically displaceable in unison with the mount along the arm 18 in directions indicated by the arrow Z, when the mount similarly is displaced in directions indicated by the arrow Z along the groove 44.

The compression plate 34 has an opening 48 defined therein near a chest wall 46 of the examinee 20, for allowing the biopsy hand assembly 38 to remove a tissue sample. The biopsy hand assembly 38 comprises a post 50 fixedly mounted on the compression plate 34, a first arm 52 having one end thereof pivotally supported on the post 50, and which is angularly movable about the post 50 along the surface of the compression plate 34, and a second arm 54 having one end thereof pivotally supported on another end of the first arm 52, and which is angularly movable about the other end of the first arm 52 along the surface of the compression plate 34. A biopsy needle 56 is mounted on the other end of the second arm 54 for movement in directions indicated by the arrow Z.

The biopsy needle 56 has a sampler 58 disposed near the lower end thereof, for sampling under suction a tissue (e.g., calcified tissue) from the biopsy region 36, which forms a lesion area (e.g., calcified area) of the breast 22. The sampler 58 of the biopsy needle 56 can be moved to a position in the vicinity of the biopsy region 36 when the first arm 52 and the second arm 54 of the biopsy hand assembly 38 are moved in an X-Y plane parallel to the surface of the compression plate 34, and when the biopsy needle 56 is moved in directions indicated by the arrow Z.

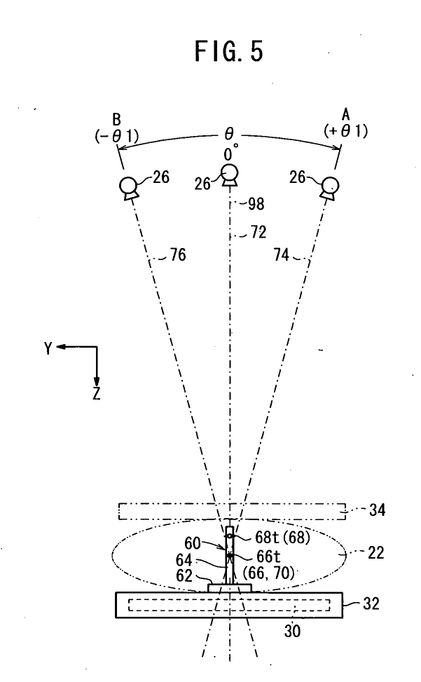

Additionally, with the mammography apparatus 12, the radiation source housing unit 28 is turned about the hinge 42, such that the radiation source 26 is placed at two positions (e.g., two angles from among the three rotational angles 0°, +θ1 and −θ1 shown in FIG. 5) and image capturing is performed at such positions, whereby a stereographic image capturing process can be performed for obtaining a radiographic image (two radiographic images) of the breast at two respective imaging angles (stereoscopic angles).

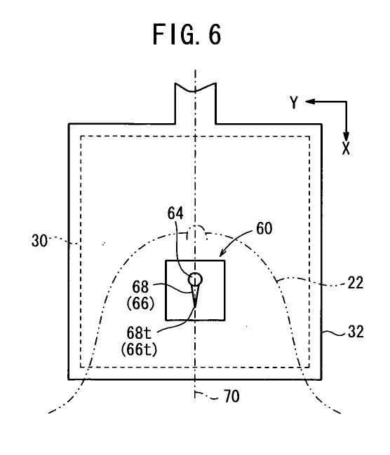

More specifically, by rotating the radiation source housing unit 28 about the hinge 42, the radiation source 26 is disposed at a substantially central portion in the height direction of the breast 22 as viewed from the side in FIG. 4, as well as at a substantially central portion of the breast 22 in front view (as viewed frontally) in FIG. 5. Moreover, the radiation source 26 is rotated in the direction of the angle θ about the axis of the center of rotation (position of the center of rotation) 70, which is disposed roughly in a center portion in the lateral direction of the breast 22 in plan view (as viewed in plan) in FIG. 6.

Accordingly, when a straight line interconnecting the position of the radiation source 26 at 0° and the central axis of rotation 70 is taken as a central axis 72, a straight line interconnecting the position A of the radiation source 26 at +θ1 and the central axis of rotation 70 is taken as a central axis 74, and a straight line interconnecting the position B of the radiation source 26 at −θ1 and the central axis of rotation 70 is taken as a central axis 76, and under a condition in which the radiation source 26 is arranged at two angles from among the three stereoscopic angles (0°, +θ1, −θ1) shown in FIG. 5, by applying radiation 24 along the central axes 72, 74, 76 and toward the central axis of rotation 70 (the breast 22), radiation 24 that passes through the breast 22 can be detected as radiation images by the solid-state detector 30 of the image capturing base 32.

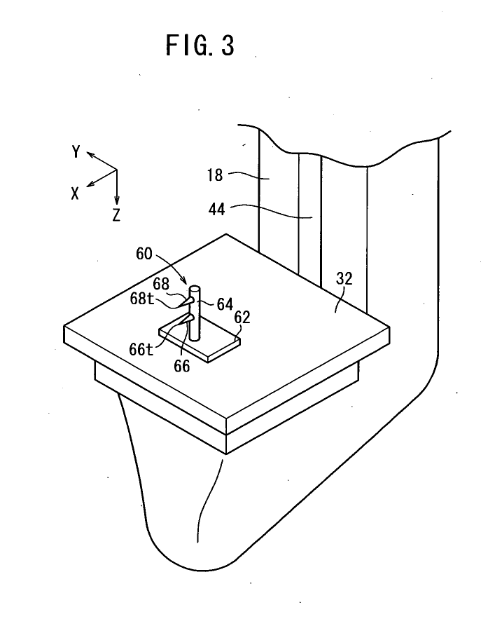

Incidentally, with the mammography apparatus 12, prior to taking a stereographic image of the breast 22, a jig 60 shown in FIGS. 3 to 6 is arranged on the image capturing base 32 and a stereographic image of the jig 60 is taken.

The jig 60 is constructed from a base plate 62 capable of being arranged on the image capturing base 32, a rod 64 which is attached to an upper surface of the base plate 62 along the Z-direction, and needle members 66, 68 that are affixed respectively at different heights on the rod 64.

The base plate 62 is made of a material that is permeable to radiation 24, whereas the rod 64 and the needle members 66, 68 are made from a material that is impervious to radiation 24, or from a material capable of absorbing radiation 24. More preferably, among the components that make up the jig 60, at least the needle members 66, 68 are made from a material impervious to radiation 24, or from a material capable of absorbing radiation 24.

Further, the end of the needle member 66, which is affixed at an intermediate portion of the rod 64, forms a reference point 66t, and the end of the needle member 66, which is affixed at the upper end of the rod 64, forms an auxiliary reference point 68t.

Concerning placement of the jig 60, the compression plate 34 is moved upwardly along the groove 44, or the compression plate 34 is taken out from the groove 44, and in a state in which the breast 22 is not arranged thereon, the jig 60 is arranged on the image capturing base 32.

At this time, the base plate 62 is arranged at a predetermined location on the image capturing base 32, such that the reference point 66t and the auxiliary reference point 68t are positioned within an area where the breast 22 will be compressed and secured between the compression plate 34 and the image capturing base 32. Stated otherwise, the jig 60 is arranged on the image capturing base 32 so that the reference point 66t and the auxiliary reference point 68t enter into the application range (radiation field) of the radiation 24.

At this time, the needle member 66 is positioned on the central axis of rotation 70, and together therewith, the reference point 66t is arranged at a center position where the breast 22 is compressed and secured. On the other hand, the needle member 68 is arranged at a position slid upwardly from the reference point 66t along the central axis 72, i.e., further upwardly than the needle member 66 that lies on the central axis 72 as viewed from the front in FIG. 5, and at a position on the central axis of rotation 70 as viewed in plan in FIG. 6.

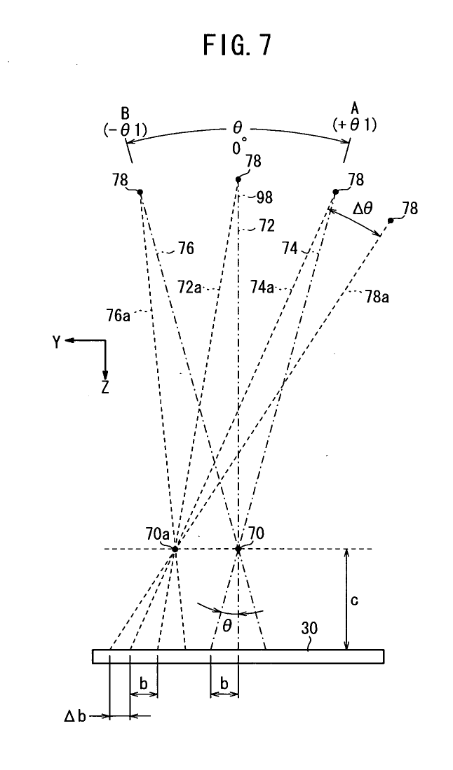

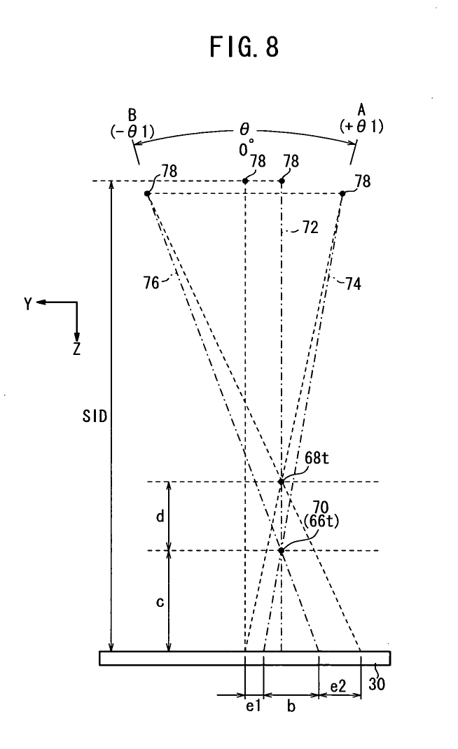

Next, the significance of disposing the reference point 66t and the auxiliary reference point 68t in the aforementioned manner shall be explained with reference to FIGS. 7 and 8.

FIG. 7 is a design drawing that illustrates schematically a stereographic image capturing process, in a case where a focal point 78 of the radiation source 26, which was intended to be arranged at the position A of +θ1, actually is arranged at a position shifted Δθ from +θ1, as a result of variances in assembly or manufacturing variances of the mammography apparatus 12.

In this case, in terms of design, it would be acceptable if radiation 24 were applied from the focal point 78 of the radiation source 26 toward the central axis of rotation 70 along the central axes 72, 74, 76 as shown by the one-dot-dash lines. However, in actuality, because the focal point 78 at the position A is shifted by Δθ from +θ1, the radiation source 26 causes the radiation 24 to be applied along the straight lines (central axes 72a, 76a, 78a) shown by the dashed lines.

More specifically, as a result of shifting of the focal point 78 at the position A by Δθ from +θ1, the position of the central axis of rotation 70 also becomes shifted to a position (central axis of rotation 70a) along the direction of the arrow Y.

A distance c is defined along the Z-direction between the central axis of rotation 70 and the solid-state detector 30, and a resultant angle θ is defined by the central axis 72 and the central axis 74. When radiation 24 is applied respectively from the 0° position and from the position A along the central axes 72, 74 toward the central axis of rotation 70, then assuming that an interval between projection images of the central axis of rotation 70 projected in the two radiation images is defined by a distance b, then the relation b=c×tan θ results.

On the other hand, when radiation 24 is applied respectively from the 0° position and from the position A along the central axes 72a, 74a toward the central axis of rotation 70a, the interval between projection images of the central axis of rotation 70a projected in the two radiation images also is defined by the distance b.

Furthermore, the shifted amount (distance Δb) of the distance b as a result of shifting of the focal point 78 at the position A by Δθ from +θ1, and from the resultant angle Δθ defined by the central axis 74a and the central axis 78a, becomes Δb=c×tan Δθ.

In this case, the angle θ or the distance b can easily be determined assuming that the positional relationship between the focal point 78, the central axis of rotation 70, and the solid-state detector 30 correspond to the design values thereof. On the other hand, the distance Δb can be determined from the actual radiation images, which are obtained by the stereographic image capturing process.

Accordingly, by disposing a reference point at a position (a position having the same height as the central axis of rotation 70) upwardly from the solid-state detector 30 by the distance c, and carrying out stereographic image capturing, the angle Δθ can be calculated using the angle θ and the distances b and Δb. As a result, a shift in the stereoscopic angle is capable of being corrected.

Consequently, with the present invention, by applying this concept, by disposing the reference point 66t on the central axis of rotation 70, and together therewith, by disposing the auxiliary reference point 68t upwardly (in the Z-direction) from the central axis of rotation 70 and the reference point 66t by a given distance d, and then applying radiation 24 to the reference point 66t and the auxiliary reference point 68t and carrying out stereographic image capturing to obtain two radiographic images, even if shifting of the stereoscopic angle is generated caused by variances in assembly or manufacturing variances of the mammography apparatus 12, the three dimensional position of the biopsy region 36 can be determined with good precision. In FIG. 8, the SID (source to image distance) is defined by a distance between the focal point 78 of the radiation source 26 and the solid-state detector 30 along the Z-direction.

When radiation 24 is applied with respect to the reference point 66t and the auxiliary reference point 68t from the radiation source 26, which is disposed at the position A, the reference point 66t and the auxiliary reference point 68t are projected in the radiation image converted from the radiation 24, and are imaged therein as projected images separated by a given distance e1. Further, when radiation 24 is applied with respect to the reference point 66t and the auxiliary reference point 68t from the radiation source 26, which is positioned at the position B, the reference point 66t and the auxiliary reference point 68t are projected in the radiation image converted from the radiation 24, and are imaged therein as projected images separated by a given distance e2.

Accordingly, if the position of the radiation source 26 is arranged at position A and position B shown in FIG. 5, when radiation 24 is applied with respect to the reference point 66t and the auxiliary reference point 68t from position A and position B, then e1=e2. On the other hand, in the case that the radiation source 26 becomes shifted from position A and position B, or if the central axis of rotation 70 becomes shifted from the central axis 72, then e1≠e2. Owing thereto, with the present embodiment, based on the ratio of the distances e1 and e2, amounts by which the positions and/or the angles are shifted are determined, and based on such determined amounts, among the three dimensional coordinate positions of the biopsy region 36, a positional shift amount thereof in the X-Y plane is corrected.

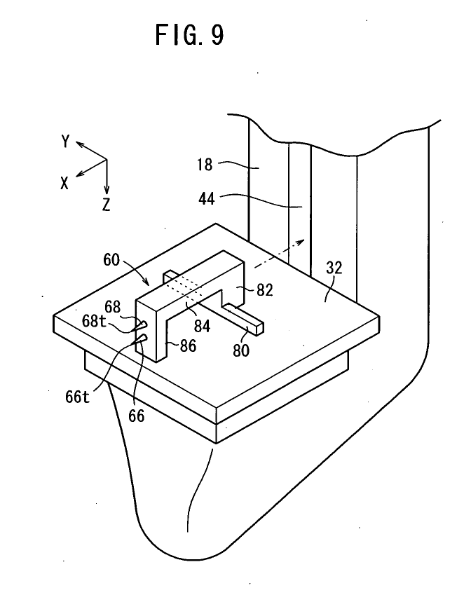

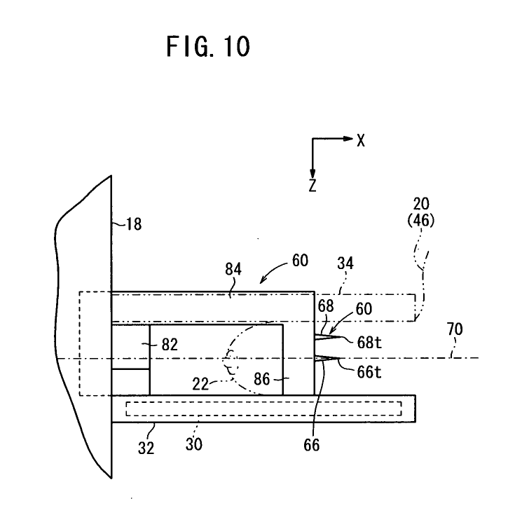

Instead of the construction shown in FIGS. 3 through 6, the jig 60 may be constructed as shown in FIGS. 9 and 10.

The jig 60 shown in FIGS. 9 and 10 is constituted from a rod shaped member 80 that extends along the Y-direction, an upstanding portion 82 that extends upwardly from a central region of the rod shaped member 80, a bridge 84 that extends in the X-direction from the upstanding portion 82, a mount 86 that is suspended downward in the Z-direction from an end of the bridge 84, and needle members 66, 68 that are affixed to a side portion on the X-direction side of the mount 86.

In this case, the upstanding portion 82 is formed so as to expand toward the groove 44 from the rod shaped member 80. Further, a width in the Y-direction of the upstanding portion 82 is shaped to substantially coincide with the width of the groove 44. Accordingly, by fitting a part of the upstanding portion 82 into the groove 44 such that the rod shaped member 80 is brought into abutment against the arm 18, the needle members 66, 68 can be arranged within a region where the breast 22 is to be compressed and secured, while the needle member 66 and the reference point 66t can be arranged on the central axis of rotation 70.

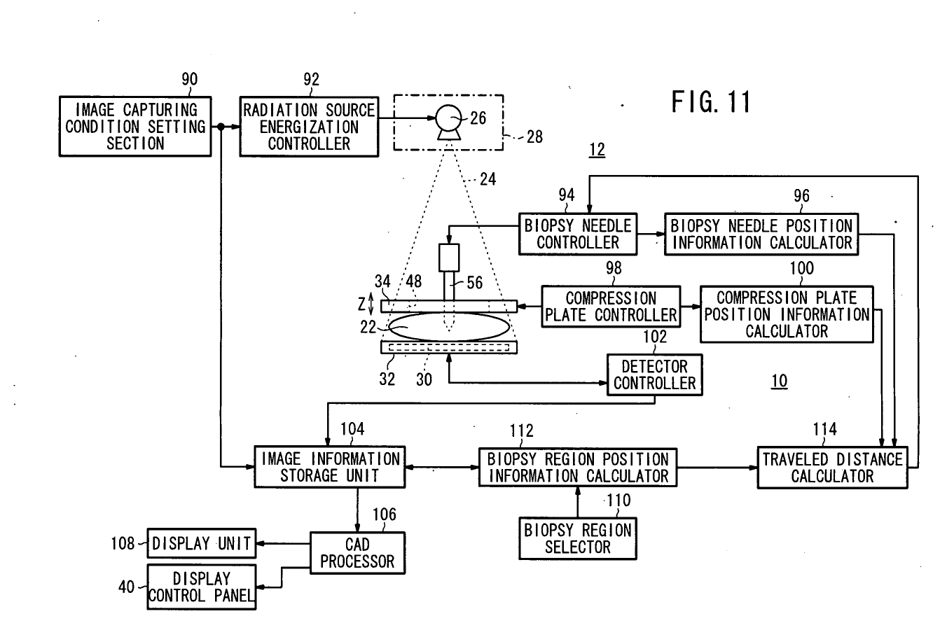

FIG. 11 shows a block diagram of the mammography apparatus 12.

As shown in FIG. 11, the mammography apparatus 12 includes an image capturing condition setting section 90, a radiation source energization controller 92, a biopsy needle controller 94, a biopsy needle position information calculator 96, a compression plate controller 98, a compression plate position information calculator 100, a detector controller 102, an image information storage unit (memory unit) 104, a CAD (Computer Aided Diagnosis) processor 106, a display unit 108, a biopsy region selector 110, a biopsy region position information calculator (position calculating unit) 112, and a traveled distance calculator 114.

Within the mammography apparatus 12, the biopsy hand assembly 38, the biopsy needle 56, the opening 48, the biopsy needle controller 94, the biopsy needle position information calculator 96, the biopsy region selector 110, and the traveled distance calculator 114 collectively make up the biopsy apparatus 10. More specifically, by assembling the biopsy apparatus 10 including these structural components, which are incorporated together in the mammography apparatus 12, a tissue of the biopsy region 36 is capable of being sampled.

The image capturing condition setting section 90 sets image capturing conditions including a tube current and a tube voltage, an application dose and an application time of the radiation 24, and the stereoscopic angle, etc. The radiation source energization controller 92 controls energization of the radiation source 26 according to the image capturing conditions. The biopsy needle controller 94 controls the biopsy hand assembly 38 (see FIGS. 1 and 2) in order to move the biopsy needle 56 to a desired position. The compression plate controller 98 moves the compression plate 34 in the directions indicated by the arrow Z. The detector controller 102 controls the solid-state detector 30 in order to store radiographic images, which are converted by the solid-state detector 30 from the radiation 24, in the image information storage unit 104.

As discussed above, because the mammography apparatus 12 performs stereographic image capturing, two radiographic images of the breast 22 taken at two stereoscopic angles, and two radiographic images of the jig 60 taken at two stereoscopic angles are stored in the image information storage unit 104. In this case, the detector controller 102 reads out the two stereoscopic angles corresponding to the two radiographic images from the image capturing condition setting section 90, and the two stereoscopic angles are stored together with the two radiographic images in the image information storage unit 104.

The CAD processor 106 processes the two radiographic images stored in the image information storage unit 104, and displays the processed radiographic images on the display unit 108 and the display control panel 40.

The biopsy region selector 110 comprises a pointing device such as a mouse or the like. Using the pointing device as the biopsy region selector 110, a doctor or radiological technician in charge, who has seen the displayed contents, i.e., the two radiographic images, on the display unit 108 and/or the display control panel 40, can select one out of a plurality of biopsy regions 36 displayed in the two radiographic images from which a tissue is to be removed. More specifically, the doctor or radiological technician selects a biopsy region 36 in one of the two radiographic images, and also selects the corresponding biopsy region 36 in the other of the two radiographic images.

First, the biopsy region position information calculator 112 reads out the two radiographic images in which the jig 60 is projected, calculates distances e1, e2 between the reference point 66t and the auxiliary reference point 68t within the two radiographic images, and from the ratio of the calculated distances e1, e2, calculates an amount of angular shift from the set value of the stereoscopic angle, and an amount of shift in position from the set value of the central axis of rotation 70. Next, The biopsy region position information calculator 112 calculates the three-dimensional position of the selected biopsy region 36 based on positions of the selected biopsy region 36 in the two radiographic images, and using the angular shift amount and the positional shift amount, corrects the positional component thereof in the X-Y plane from among the three dimensional coordinate positions. The three-dimensional position of the selected biopsy region 36 can be calculated according to a known three-dimensional position calculating scheme implemented in the stereographic image capturing process.

The biopsy needle position information calculator 96 calculates position information of the tip end of the biopsy needle 56. When a tissue is sampled from the biopsy region 36, the biopsy needle position information calculator 96 calculates the position of the tip end of the biopsy needle 56 before tissue is sampled from the biopsy region 36. Stated otherwise, the position of the tip end of the biopsy needle 56 is calculated before the biopsy needle 56 pierces the breast 22.

The compression plate position information calculator 100 calculates position information of the compression plate 34, which has been moved with respect to the image capturing base 32 by the compression plate controller 98. Since the compression plate 34 presses against the breast 22 with respect to the image capturing base 32 and holds the breast 22 in a pressed state, the position information of the compression plate 34 represents thickness information of the breast 22 as the breast 22 is being pressed.

The traveled distance calculator 114 calculates the distance by which the biopsy needle 56 is moved with respect to the biopsy region 36, based on the three-dimensional position of the biopsy region 36, which has been calculated and corrected by the biopsy region position information calculator 112, the position of the tip end of the biopsy needle 56, which has been calculated by the biopsy needle position information calculator 96, and the position of the compression plate 34 (i.e., the thickness of the breast 22), which has been calculated by the compression plate position information calculator 100. Based on the distance calculated by the traveled distance calculator 114 that the biopsy needle 56 has moved with respect to the biopsy region 36, the biopsy needle controller 94 moves the biopsy needle 56 in order to enable a tissue to be sampled from the biopsy region 36.

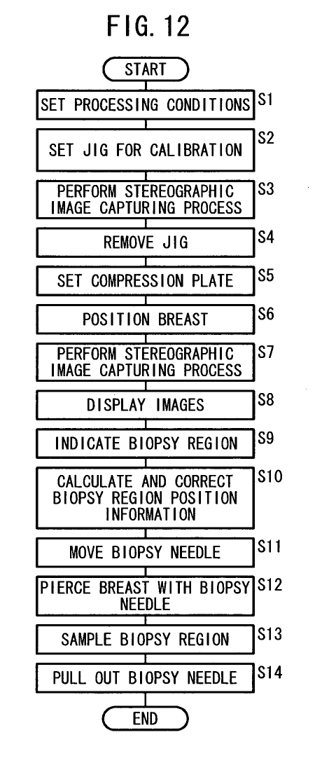

The mammography apparatus 12 according to the present invention is constructed basically as described above. Next, operations of the mammography apparatus 12 (radiographic image capturing method, position calculating method) shall be described below with reference to the flowchart shown in FIG. 12.

Herein, a case shall be explained in which the compression plate 34 first is removed from the groove 44 when a stereographic image is captured with respect to the jig 60, and then after a stereographic image thereof has been captured, the compression plate 34 is installed in the groove 44 and a stereographic image capturing process is carried out with respect to the breast 22 of a subject.

In step S1, initially, the image capturing condition setting section 90 (see FIG. 11) sets image capturing conditions, including a tube current and a tube voltage of the radiation source 26, an application dose and an application time for the radiation 24, a stereoscopic angle of the radiation source 26, etc., depending on conditions of the breast 22. The image capturing conditions are set in this manner in the radiation source energization controller 92.

Next, in step S2, the jig 60 is disposed on the image capturing base 32 in the position shown in FIGS. 4 to 6, such that the reference point 66t is arranged on the central axis of rotation 70, while both the reference point 66t and the auxiliary reference point 68t are arranged on the central axis 72.

In step S3, the mammography apparatus 12 drives the radiation source 26 and carries out stereographic image capturing with respect to the jig 60. In this case, the radiation source housing unit 28 is rotated about the hinge 42 (see FIG. 1) so that, for example, by positioning the radiation source 26 respectively at the positions A and B shown in FIG. 5, and applying radiation 24 therefrom, radiation 24 that has permeated the jig 60 is detected as radiographic images by the solid-state detector 30 provided in the image capturing base 32.

In this case, because the needle members 66, 68 are made from a material impervious to radiation 24 or from a material that absorbs radiation 24, as shown in FIG. 8, in each of the radiographic images at the positions A and B, the reference point 66t and the auxiliary reference point 68t are projected respectively, and distances between each of the projection images of the reference point 66t and the auxiliary reference point 68t are designated as e1 and e2, respectively.

The detector controller 102 controls the solid-state detector 30 and two radiographic images are obtained. These two radiographic images are stored in the image information storage unit 104 together with the stereoscopic angles at each of the positions A and B as read out from the image capturing condition setting section 90. Additionally, the CAD processor 106 carries out image processing with respect to the two stereographic images that are stored in the image information storage unit 104, and after such image processing, displays the two radiographic images on the display unit 108 and the display control panel 40. Consequently, a doctor or radiological technician can confirm easily that stereographic image capturing with respect to the jig 60 has been completed.

In step S4, a doctor or radiological technician removes the jig 60 from the image capturing base 32, and next, in step S5, fits the compression plate 34 into the groove 44.

Next, in step S6, the doctor or radiological technician positions the breast 22 of the examinee 20. More specifically, the breast 22 is placed in a predetermined position (facing the opening 48), and thereafter, the compression plate controller 98 moves the compression plate 34 toward the image capturing base 32 in the direction indicated by the arrow Z, thereby compressing and positioning the breast 22 against the image capturing base 32.

As a result, the breast is compressed and secured between the image capturing base 32 and the compression plate 34. The compression plate position information calculator 100 calculates position information of the compression plate 34 with respect to the image capturing base 32, and outputs the calculated position information to the traveled distance calculator 114.

After the above preparatory process for stereographic image capturing of the breast 22 is completed, in step S7, the mammography apparatus 12 energizes the radiation source 26 again, this time in order to perform a stereographic image capturing process on the breast 22. In this case, the radiation source housing unit 28 is turned about the hinge 42 (see FIG. 1) to position the radiation source 26 respectively at the positions A and B, and to apply radiation 24 therefrom. Radiation 24 that passes through the breast 22 is applied to the solid-state detector 30 in the image capturing base 32, and the radiation 24 is detected as radiographic images.

The detector controller 102 controls the solid-state detector 30, whereby two radiographic images are obtained. These two radiographic images are stored in the image information storage unit 104 together with the stereoscopic angles at each of the positions A and B, which are read out from the image capturing condition setting section 90.

In step S8, the CAD processor 106 processes the two radiographic images of the breast 22 that are stored in the image information storage unit 104, and displays the two processed radiographic images on the display unit 108 and the display control panel 40.

In step S9, using the biopsy region selector 110, which is a pointing device such as a mouse or the like, a doctor or radiological technician selects, from among a plurality of biopsy regions 36 in the two radiographic images displayed on the display unit 108 and/or on the display control panel 40, a biopsy region 36 from which a tissue sample will be removed.

In step S10, when the biopsy region 36 has been selected, first, the biopsy region position information calculator 112 reads out the two radiographic images in which the jig 60 has been captured and calculates distances e1, e2 between the reference point 66t and the auxiliary reference point 68t within the two radiographic images, and from the ratio of the calculated distances e1, e2, calculates an amount of angular shift from the set value of the stereoscopic angle, and an amount of shift in position from the set value of the central axis of rotation 70. Next, The biopsy region position information calculator 112 calculates the three-dimensional position of the biopsy region 36 based on positions of the biopsy region 36 in the two radiographic images of the breast 22, and using the angular shift amount and the positional shift amount, corrects the positional component thereof in the X-Y plane from among the three dimensional coordinate positions.

On the other hand, the biopsy needle position information calculator 96 calculates the position of an end of the biopsy needle 56 before the biopsy needle 56 pierces the breast 22.

Accordingly, the traveled distance calculator 114 calculates the distance by which the biopsy needle 56 is moved with respect to the biopsy region 36, based on the three-dimensional position of the biopsy region 36, which has been calculated and corrected by the biopsy region position information calculator 112, the position of the tip end of the biopsy needle 56, which has been calculated by the biopsy needle position information calculator 96, and the position of the compression plate 34, which has been calculated by the compression plate position information calculator 100.

In step S11, based on the distance calculated from the traveled distance calculator 114, the biopsy needle controller 94 moves the biopsy needle 56 in order to enable a tissue to be sampled from the biopsy region 36. As a result, the biopsy hand assembly 38 moves the first arm 52 and the second arm 54 in the X-Y plane to position the biopsy needle 56 at a position confronting the biopsy region 36 (i.e., a predetermined position along the Z-direction with respect to the biopsy region 36). Next, in step S12, the biopsy needle 56 is moved in the Z-direction, and the biopsy needle 56 pierces the breast 22 through the opening 48 formed in the compression plate 34.

In step S13, when the sampler 58 of the biopsy needle 56 has reached a position near the biopsy region 36, the biopsy needle 56 starts to sample tissue from the biopsy region 36 under suction. Thereafter, in step S14, the biopsy needle 56 is moved in the Z-direction until the biopsy needle 56 is pulled out from the breast 22, whereupon the tissue sampling operation is brought to an end.

As described above, with the mammography apparatus 12 of the present embodiment, because the reference point 66t is positioned removably by the jig 60 on the central axis of rotation 70 of the radiation source 26, when a stereographic image is captured with respect to the reference point 66t, the reference point is disposed on the central axis of rotation 70 and stereographic image capturing is carried out. On the other hand, when a stereographic image is captured with respect to the breast 22, after the jig 60 equipped with the reference point 66t has been removed, the breast 22 is positioned on the image capturing base 32 and stereographic image capturing of the breast 22 may be performed.

Consequently, in radiographic images obtained from each of the stereographic image capturing processes, because either an image in which the reference point 66t is reflected therein is produced, or alternatively, an image having the breast 22 is included therein is produced, the reference point 66t is not reflected within the breast 22. Accordingly, a doctor or radiological technician, while observing the two radiographic images in which the breast 22 is imaged, can easily perform positioning of the biopsy apparatus 10 in the biopsy region 36 from which tissue is to be sampled, or alternatively, can easily carry out positioning in order to move the breast 22 to a suitable position.

Further, since the reference point 66t is not present when a stereographic image is taken with respect to the breast 22, the applied area (radiation field) of the radiation 24 can be limited to within a minimum necessary range, and thus needless and excessive exposure of the examinee 20 to radiation can be avoided.

Further, a three dimensional position of the reference point 66t is calculated based on two radiographic images obtained by carrying out stereographic image capturing with respect to the reference point 66t, while on the other hand, a three dimensional position of the biopsy region 36 inside the breast 22 is calculated based on two radiographic images obtained by carrying out stereographic image capturing with respect to the breast 22. Accordingly, even if there are errors in the three dimensional position of the biopsy region 36 caused by shifting of the stereoscopic angle, the three dimensional position of the biopsy region 36 can be corrected using the three dimensional position of the reference point 66t.

In this manner, compared to the disclosures of Japanese Laid-Open Patent Publication No. 2002-528220 (PCT) and Japanese Laid-Open Patent Publication No. 2003-024321, with the present invention, the number of times that stereographic image capturing is carried out is increased by one. However, even though stereographic image capturing is not performed simultaneously with respect to the reference point 66t and the breast 22, any errors in the three dimensional position of the biopsy region 36 caused by shifting of the stereoscopic angle can easily be corrected.

In addition, in the mammography apparatus 12, by arranging the jig 60, which is equipped with the reference point 66t, removably with respect to the image capturing base 32 that accommodates the solid-state detector 30, setting of the reference point 66t on the central axis of rotation 70 can easily be carried out.

In this case, in a state in which the jig 60 is arranged on the image capturing base 32, and after the radiation source 26 has applied radiation 24 with respect to the reference point 66t from two stereoscopic angles, the jig 60 is removed from the image capturing base 32. Then, in a state in which the jig 60 has been removed from the image capturing base 32 and the breast 22 has been compressed and secured between the compression plate 34 and the image capturing base 32, since the radiation source 26 applies radiation 24 from two stereoscopic angles with respect to the breast 22, errors in the three dimensional position of the biopsy region 36 can be corrected effectively.

Additionally, as viewed from the front in FIG. 5, the central axis of rotation 70 and the reference point 66t are disposed at a central position within the image of the breast 22, which is compressed and secured between the compression plate 34 and the image capturing base 32. Further, as viewed in plan in FIG. 6, the central axis of rotation 70 and the reference point 66t are disposed at a central position within the image of the breast 22, which is compressed and secured between the compression plate 34 and the image capturing base 32. Owing thereto, errors in the three dimensional position of the biopsy region 36 caused by shifting of the stereoscopic angle can be corrected accurately.

Further, by additionally providing the auxiliary reference point 68t on the jig 60, the three dimensional position of the biopsy region 36 is capable of being corrected with greater precision.

More specifically, in the image information storage unit 104, on the one hand, two radiographic images obtained by stereographic image capturing with respect to the jig 60 are stored in the image information storage unit 104 together with two stereoscopic angles corresponding to such stereographic image capturing, while on the other hand, two radiographic images obtained by stereographic image capturing with respect to the breast 22 are stored in the image information storage unit 104 together with two stereoscopic angles corresponding to such stereographic image capturing. When the three dimensional position of the biopsy region 36 is calculated in the biopsy region position information calculator 112, initially, the two radiographic images and the two stereoscopic angles of the jig 60 are read out from the image information storage unit 104, the distances e1, e2 in the two radiographic images are calculated, and the amount of angular shift from set values of the stereographic angles, as well as the amount of shift in position from the set value of the central axis of rotation 70, are calculated from the ratio of the calculated distances e1, e2. Based thereon, among the three dimensional coordinate positions of the biopsy region 36, which were obtained based on the position of the biopsy region 36 in the two radiographic images of the breast 22, the position component in the X-Y plane (direction) is corrected using the angular shift amount and the amount of shift in position. As a result, the three dimensional position of the biopsy region 36 can be corrected with good efficiency.

Although certain preferred embodiments of the present invention have been shown and described in detail, it should be understood that various changes and modifications may be made to the embodiments without departing from the scope of the invention as set forth in the appended claims.

What is claimed is:

1. A radiographic image capturing apparatus comprising:
a radiation source that outputs radiation;
a radiation detector for detecting the radiation and converting the radiation into a radiographic image; and
a reference point, which is disposed in a removable manner between the radiation source and the radiation detector,
wherein the reference point is disposed at a position of a center of rotation of the radiation source, which is set between the radiation source and the radiation detector, and
wherein, by rotating the radiation source about the position of the center of rotation, the radiation source applies the radiation with respect to the reference point from at least two different angles.

2. A radiographic image capturing apparatus according to claim 1, further comprising an image capturing base accommodating the radiation detector therein,
wherein the reference point is disposed on a jig, which is arranged removably on the image capturing base.

3. A radiographic image capturing apparatus according to claim 2, further comprising a compression plate, which is displaceable toward the image capturing base to compress and secure an object to be examined of a subject on the image capturing base, in case that the object to be examined is positioned on the image capturing base,
wherein, in a state in which the jig is arranged on the image capturing base, and after the radiation source has applied the radiation with respect to the reference point from the two angles, the jig is removed from the image capturing base, and
wherein, in a state in which the jig has been removed from the image capturing base and the object to be examined has been compressed and secured between the compression plate and the image capturing base, the radiation source applies the radiation with respect to the object to be examined from the two angles.

4. A radiographic image capturing apparatus according to claim 3, wherein, in front view, the position of the center of rotation and the position of the reference point are disposed at a predetermined position inside the object to be examined, which is compressed and secured between the compression plate and the image capturing base.

5. A radiographic image capturing apparatus according to claim 3, wherein, in plan view, the reference point is disposed at a center position inside the object to be examined, which is compressed and secured between the compression plate and the image capturing base.

6. A radiographic image capturing apparatus according to claim 3, further comprising:
a memory unit for storing two radiographic images obtained by the radiation detector by applying the radiation with respect to the reference point, while also storing two radiographic images obtained by the radiation detector by applying the radiation with respect to the object to be examined, which has been compressed and secured; and
a position calculating unit for calculating a three dimensional position of the reference point and a three dimensional position of a biopsy region inside the object to be examined based on the radiographic images stored in the memory unit,
wherein the position calculating unit corrects the three dimensional position of the biopsy region inside the object to be examined using the three dimensional position of the reference point.

7. A radiographic image capturing apparatus according to claim 6, wherein:
in case that the jig is arranged on the image capturing base, an auxiliary reference point is disposed on the jig, the auxiliary reference point being arranged on a vertical axis, which is orthogonal to the radiation detector and passes through the reference point and the position of the center of rotation;
the radiation source applies the radiation with respect to the reference point and the auxiliary reference point from the two angles in case that the jig is arranged on the image capturing base;
the memory unit stores two radiographic images obtained by the radiation detector by applying the radiation with respect to the reference point and the auxiliary reference point; and
the position calculating unit calculates a three dimensional position of the reference point, a three dimensional position of the auxiliary reference point, and a three dimensional position of the biopsy region based on the radiographic images stored in the memory unit, and within the three dimensional position of the biopsy region, corrects a position in a direction along the radiation detector based on the calculated three dimensional position of the reference point and the calculated three dimensional position of the auxiliary reference point.

8. A radiographic image capturing method comprising the steps of:
disposing a reference point in a removable manner on a position of a center of rotation of a radiation source, which is set between the radiation source and a radiation detector;
applying radiation by the radiation source with respect to the reference point from at least two different angles, by rotating the radiation source about the position of the center of rotation; and
detecting the radiation by the radiation detector, and converting the radiation into two radiographic images.

9. A position calculating method comprising the steps of:
disposing a reference point in a removable manner on a position of a center of rotation of a radiation source, which is set between the radiation source and an image capturing base;
applying radiation by the radiation source with respect to the reference point from at least two different angles, by rotating the radiation source about the position of the center of rotation;
detecting the radiation by the radiation detector, and converting the radiation into two radiographic images; and
calculating a three dimensional position of the reference point by a position calculating unit based on the two radiographic images.
